(19) United States
(12) Patent Application Publication
(10) Pub. No.: US 2021/0338363 A1
(43) Pub. Date: Nov. 4, 2021

(54) Mask For Use In Endoscopic Procedures

(71) Applicant: MASKAiON Corporation, Ridgefield, CT (US)

(72) Inventors: Ron Hadani, Even-Yahuda (IL); Yosef Krespi, New York, NY (US)

(21) Appl. No.: 17/246,323

(22) Filed: Apr. 30, 2021

Related U.S. Application Data
(60) Provisional application No. 63/019,318, filed on May 2, 2020.

(51) Int. Cl. A61B 90/00; A61B 1/00

(52) U.S. Cl. CPC A61B 90/05; A61B 1/00147

(57) ABSTRACT

There is provided a mask for use in endoscopic procedures that includes a unitary concave and bowl-shaped body portion having an inner surface and an outer surface with the body having a first opening for receiving an instrument, where the first opening in the body is defined by an edge, and the outer surface radially extends from the edge with a unitary curved wall having a peak and extending from the peak to a rim. The rim defines a second opening that is wider than the first opening and having a diameter of sufficient width to cover a patient's nose and open mouth. The mask has at least one filter positioned on the body portion to permit air to pass through the body portion, and a seal is positioned around or over the first opening to secure the instrument hermetically when received through the first opening. The filter is of a size to prevent known viruses and microbials, including coronavirus, from transmitting into the air external to the mask.

### CROSS REFERENCE TO RELATED APPLICATIONS

This application claims the benefit of and priority to U.S. Provisional Application No. 63/019,318 filed on May 2, 2020 and incorporated by reference herein in its entirety.

### FIELD OF THE INVENTION

The present invention relates to medical masks, and in particular to protection equipment for patients and medical personnel during endoscopic procedures.

### BACKGROUND

Medical professionals in the operating room are able to wear and adopt a full set of protective equipment, such as masks, gloves, visors, scrubs, and gowns. In a medical office or clinical environment, however, it is not possible for the medical profession to be fully geared up with protective equipment. This leaves the medical professional at risk of exposure to contaminants, particularly from the patient's respiratory process during the procedure. There is a need for a mask which protects the medical professional from exposure, which does not interfere with medical procedures, such as ones that are endoscopic in nature. While a risk of exposure always exists in these types of medical procedures, and the medical professional should always be protected, the risk is heightened now, during a global pandemic. Some endoscopic procedures cannot be postponed, so the medical professional performs them at great risk to themselves. This makes the need for a mask that protects the medical professional from exposure to a patient's respiratory droplets all the more pressing.

### SUMMARY

The present invention is a mask to be placed over a patient's mouth and nose during an endoscopic procedure so as to protect the medical professional and the working environment from microdroplets of liquid expelled by the patient's respiratory system during the procedure, including during normal breathing, coughing, or sneezing etc. The mask would be secured to the patient's head by a strap of some sort so the mask would not shift during the procedure.

The mask of the present invention is of a general concave outer surface with at least two openings. The first opening preferably centrally located at the peak of the concave body, for receiving an endoscope or other instrument through the mask and which would extend into the patient's mouse or nasal cavity during a medical procedure. Endoscope size typically ranges from 2 millimeters to 12 millimeters wide, so the opening is at least wide enough to accommodate the endoscope through the opening. An adjustable seal surrounds the opening so as to seal the endoscope hermetically. The second opening is larger than the first and is defined by and located at the rim of the mask, and may vary in size in order to fit a variety of patients' faces. The second opening will be the opening in contact with the patient's face, and the rim will preferably be covered in a soft material. The mask is secured to a patient's face using a strap, which is made of elastic or another suitable material. Additional openings may be included on the mask for additional instruments, such as for suction, forceps, water supply, dental or dental power tools etc. Each of the additional opening would also feature a hermetic seal.

One or more filters are positioned in the inner surface of the mask for filtration of microdroplets from the respiratory process of the patient. The filters are air permeable but would prevent any viruses and microbials from going through. Suitable filters include those in the range of 70-micron to 100-micron size or other suitable size so as to keep viruses and microbials, including human coronavirus particles, from transmitting into the external air outside the mask.

In an embodiment, there is a mask for use in endoscopic procedures which includes a unitary concave and bowl-shaped body portion having an inner surface and an outer surface with the body having a first opening for receiving an instrument. The first opening in the body is defined by an edge, with the outer surface radially extending from the edge with a unitary curved wall having a peak and extending from the peak to a rim. The rim defines a second opening which is wider than the first opening and has a diameter of sufficient width to cover a patient's nose and open mouth. The mask includes at least one filter positioned on the body portion to permit air to pass through the body portion and a seal positioned around or over the first opening to secure the instrument when received through the first opening.

In an embodiment, the mask of the present invention, the mask has a second opening which ranges in size to fit a variety of patients. In another embodiment, the mask of the present invention has a first opening which ranges in size from 2 mm to 12 mm to accommodate an endoscope or instrument. The first opening is centrally located on the mask, but other locations are within the scope. The seal is positioned around or over the first opening and seals the endoscope hermetically. The seal is preferably flexible so as to maintain the hermetic seal if and when the instrument moves during a procedure.

In an embodiment, the mask of the present invention includes the at least one filter positioned on the inner surface of the mask. The filter or filters are positioned for filtration of any microdroplets from the respiratory process of the patient. The filter or filters are of a size to prevent known viruses and microbials from transmitting into the external air outside the mask. The mask includes at least one filter that is of a size to prevent coronavirus particles from transmitting into the external air outside the mask. In an embodiment, the mask of the present invention has at least one filter is in the size range of 70 microns-100 microns. The filter or filters of the mask can be made of a porous, grid-like material.

In an embodiment, the mask of the present invention has additional, hermetically sealed openings which are included on the mask for surgical instruments in addition to the primary instrument. The mask of the present invention can be secured over the patient's nose and mouth during use, with the inner surface of the mask facing the patient. The mask can also be secured to the patient's head by the use of a strap while in use. The strap secures the mask to the patient's head is elastic or elastic material.

In an embodiment, the mask of the present invention has a bowl-shaped body portion made of a transparent material. The transparent material allows a medical professional visibility into the patient's mouth through the mask surface during a procedure. The bowl-shaped body portion may be made of a hypoallergenic material. The mask includes an embodiment where the rim is covered by a soft, flexible material capable of molding to the patients face. The mask of the present is capable of withstanding repeated sterilization processes. The mask can also be made as a single-use and disposable mask.

### BRIEF DESCRIPTION OF THE DRAWINGS

FIG. 1 is a top view of the outer surface of the present invention.

FIG. 2 is a top view of the inner surface of the present invention.

FIG. 3 is a side view of the outer surface of the present invention.

FIG. 4 is a cross section view of the present invention.

FIG. 5 is an illustration of the present invention in use, placed over a patient's face.

### DETAILED DESCRIPTION OF THE DRAWINGS

Before the disclosure is described, it is to be understood that this disclosure is not limited to the particular apparatuses and methods described below. It is also to be understood that the terminology used herein is for the purpose of describing particular embodiments only, and is not intended to be limiting.

As is used in the disclosure and the appended claims, the singular forms “a,” “an,” and “the” include plural referents unless the context clearly dictates otherwise. The methods and apparatuses disclosed herein may lack any element that is not specifically disclosed herein.

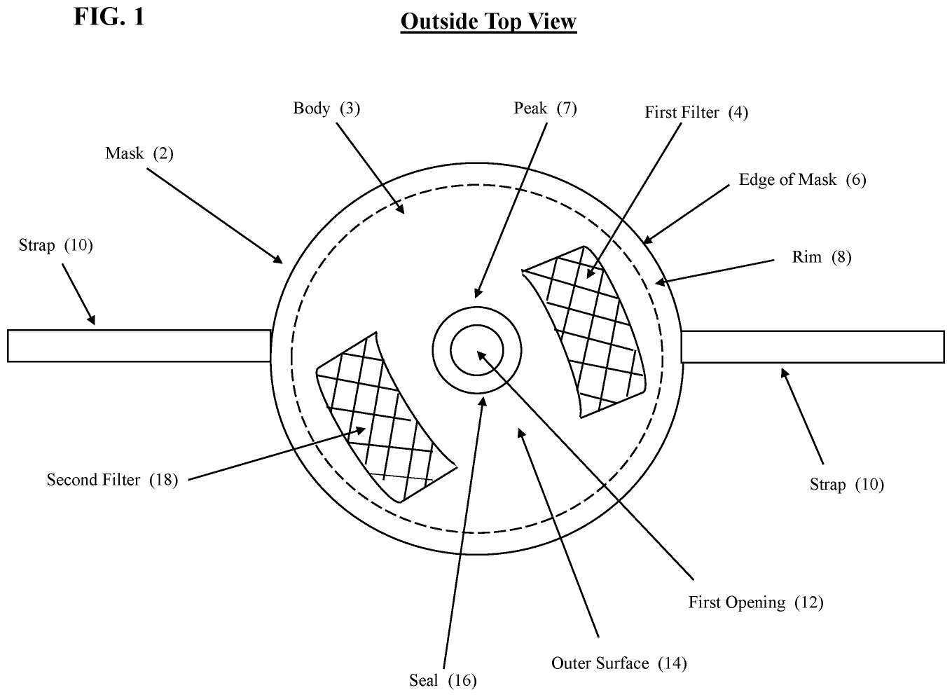

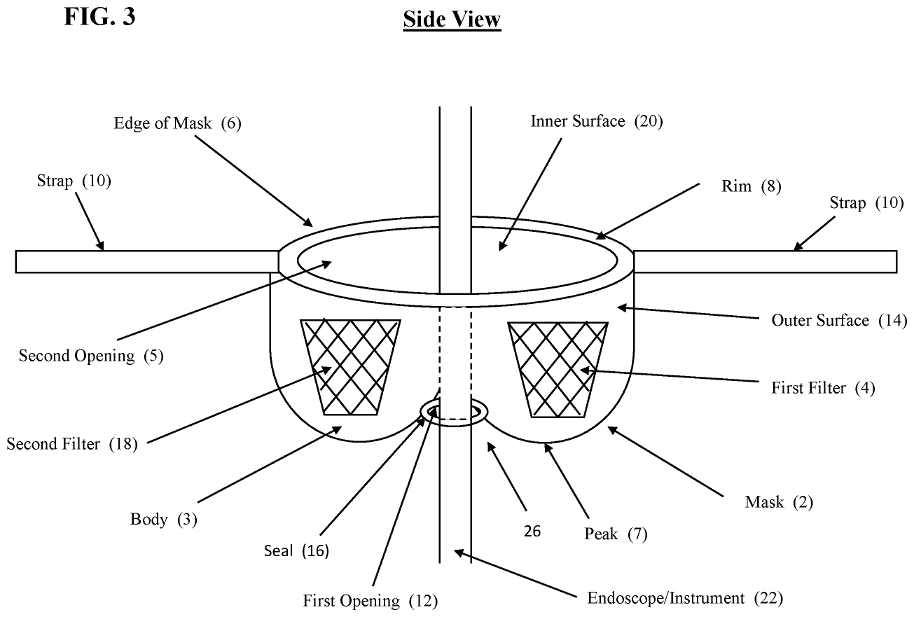

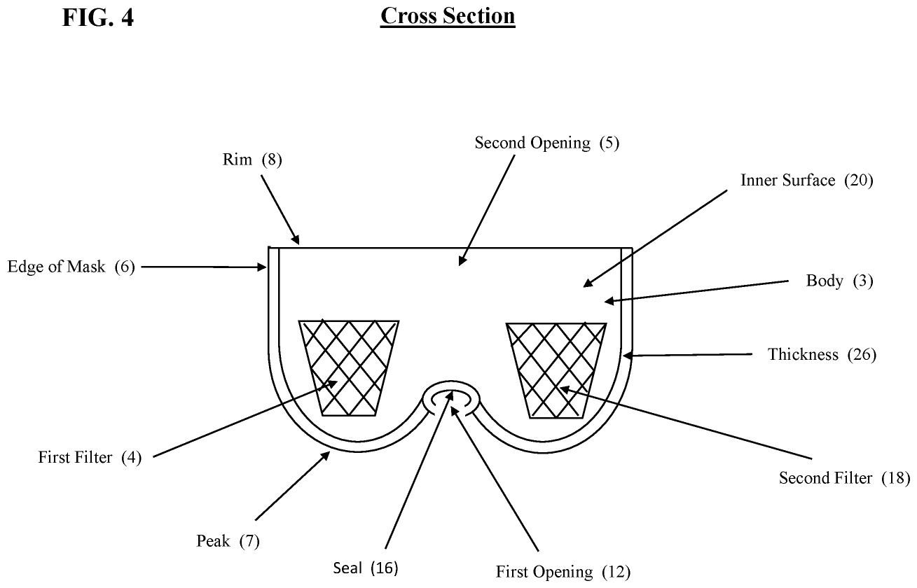

Referring to FIGS. 1 through 5, there is illustrated a mask of the present invention for use in medical procedures, especially of the endoscopic nature. The mask 2 is constructed in such a way that when used correctly, the respiratory processes of the patient 24, including the microdroplets of liquid expelled during normal breathing, coughing, and sneezing etc., are filtered from the air external to the mask 2. This protects the medical professionals carrying out an endoscopic procedure from any potential infection(s) the patient may have. The mask may be used for other medical procedures, such as dental, as well.

In the illustrated embodiment, the mask 2 includes a smooth, unitary, bowl-shaped body 3 that has two round openings. The mask 2 may be integrally formed from a single piece of material and then sized and shaped to the desired dimensions. There is a peak 7, with a unitary, curved wall with an outer surface 14 that extends radially to a rim 8. The peak 7 is formed above the first opening 12 which is located at a distance below and with the first opening forming an indented area 26 along the outer surface of the mask. Within this indented area 26, the opening 12 exists to receive a desired medical instrument 22. Stated differently, from the opening 12, the outer surface 14 of the mask 2 extends radially, uniformly, and upwardly to a peak 7 and then the outer surface 14 walls continue to extend downward from the peak 7, preferably uniformly and at a slight angle, to reach the edge 6 and rim 8 of the mask 2. The height of the mask 2 from the edge 6 and rim 8 to the peak 7 is such that the mask 2 covers the distance from and over a mouth area and covers a patient's nose when placed over a patient. A volume is thereby created inside the mask 2 which completely covers the respiratory area (nose and mouth) of the patient. The thickness 26 of the mask 2 is such that so the mask can maintain its shape while the medical professional holds it and inserts a surgical instrument through the opening in the mask. In alternative embodiments, the second opening may be other shapes, such as more triangular, so as to cover the mouth and nose of patient more narrowly. In one embodiment, body 3 is made of a sterile and hypoallergenic material. Those with ordinary skill in the art recognize that there are many materials that the body 3 can be made of that are hypoallergenic. In an alternative embodiment, the bowl is made with a transparent material so medical professionals have improved vision to the desired area of the patient and control the insertion of surgical instruments 22.

As noted above, the first opening 12 is located at or below the peak 7 of the mask 2 in an indented area and the first opening 12 is preferably centrally located. The first opening 12 is designed to accommodate a surgical instrument, which in the illustrated embodiment is an endoscope 22. In the illustrated embodiment, an endoscope 22 is inserted through the first opening 12 in the direction of outer surface 14 to inner surface 20 into the patient's 24 mouth. The mask 2 may be single use/disposable or it may be reusable and able to undergo sterilization processes.

On or around the first opening 12 is a seal 16. This seal 16 in the illustrated embodiment is positioned on or around the first opening 12 to seal the endoscope 22 hermetically into the first opening 12. The seal 16 is flexible as to allow for movement of the instrument during the medical procedure without breaking the airtight barrier. This hermetic seal helps to ensure patient 24 respiration does not escape through the first opening 12. Those with ordinary skill in the art know that instruments vary in size. In alternative embodiments, the first opening 12 can range in size in order to accommodate different sizes of instrument. In the illustrated embodiment, the first opening 12 ranges in size from 12 mm-20 mm to accommodate the typical range of endoscope sizes.

The second opening 5 is the larger of the two openings, and is the opening that comes in contact with a patient 24. The second opening 5 is large enough to cover the patient's 24 mouth and nose. In alternative embodiments, the size of the second opening 5 may vary in order to fit different patients 24. The second opening 5 is at the widest part of the mask 2, opposite of the peak 7, and begins at the edge 6 of the mask 2. The edge 6 of the mask 2 creates a rim 8. The circumference of opening 5 (or perimeter for non-circular shapes), along with the mask 2 height and width and diameter of the opening 5 form the volume to cover the patient's nose and mouth.

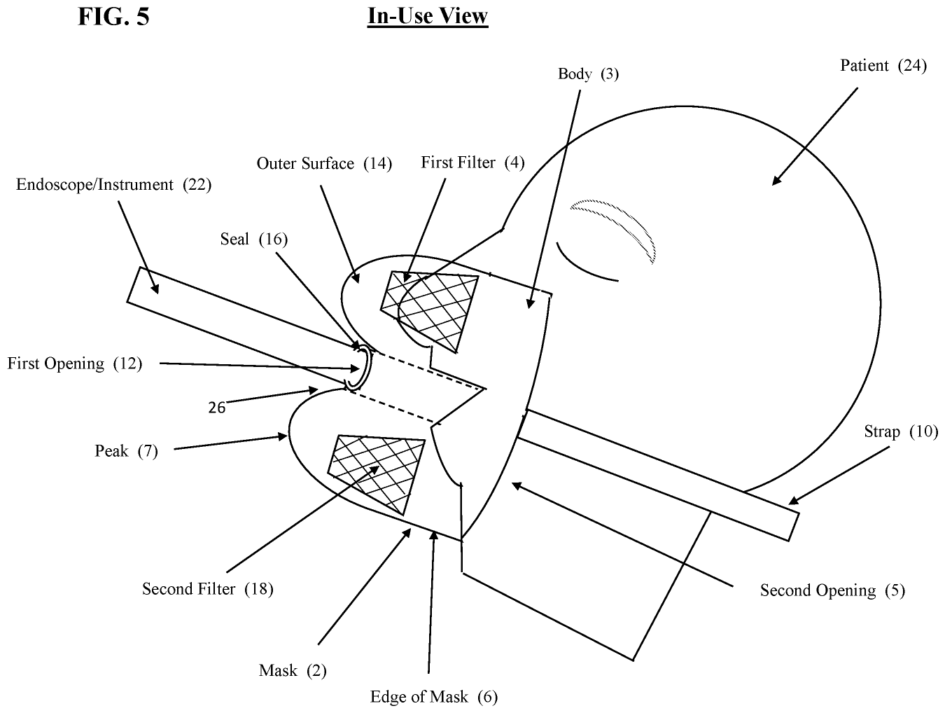

While the mask 2 is in use, the rim 8 is flush against the face of the patient 24. FIG. 5 shows the correct positioning of the mask while in use. The flush placement against the patient's 24 face is important in the mask 2 fulfilling its purpose of protecting medical professionals during endoscopic procedures. The mask 2 is secured to the patient's 24 head by the use of a strap 10. There are many materials the strap could be made out of while still achieving its purpose. The strap 10 secures the mask 2 to the patient's 24 face by means of elastic in the illustrated embodiment, but in alternative embodiments can be fastened in other suitable fashion. Fastening the mask 2 to the patient's 24 head will allow the medical professional more ease to operate, since he or she will not have to hold the mask in place. In an alternative embodiment, the rim 8 may be covered with a pad or other soft material to ensure the comfort of the patient 24. The soft covering of the rim 8 will also allow for a snug fit of the mask against the patient's face.

While the illustrated embodiment has only two openings, alternative embodiments may include additional openings for additional instruments such as for suction, forceps, dental or power or water supplied tools. These additional openings would feature a hermetic seal 16 similar to the first opening 12. Those with ordinary skill in the art understand that as the size and shape of surgical tools vary, so will the size and shape of additional openings in alternative embodiments.

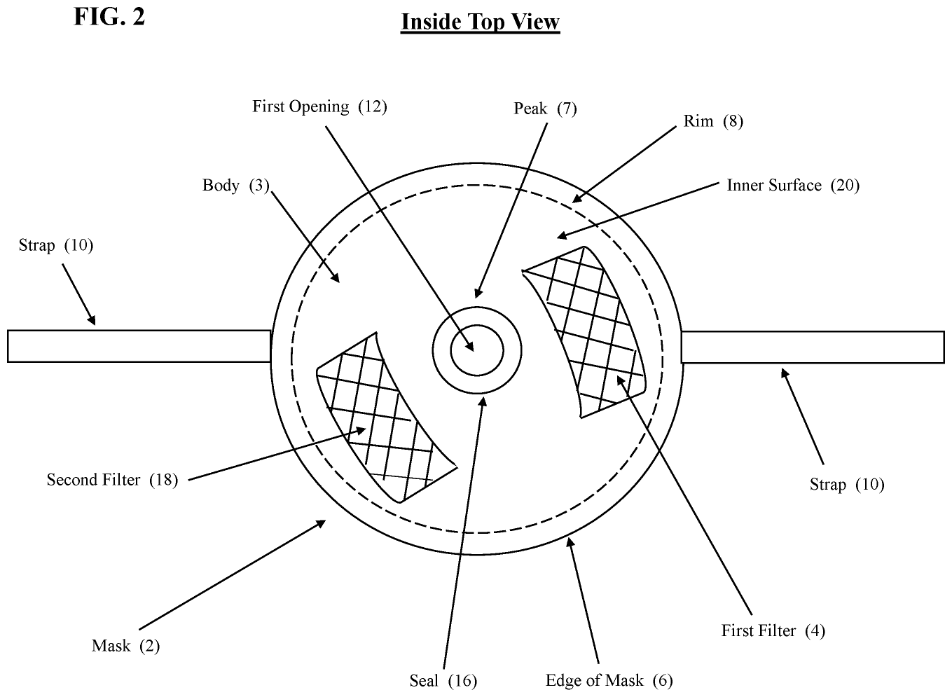

The body 3 has at least one filter. The illustrated embodiment includes a first filter 4 and a second filter 18. As shown in FIG. 1 and FIG. 2, the first and second filters 4, 18 are integrated into the body 3 of the mask 2, and can be seen when looking at either the inner surface 20 or the outer surface 14. The first and second filters 4, 18 are located on the unitary wall that extends radially from peak 7 to rim 8. In the illustrated embodiment, the first and second filters 4, 18 are made of an air-permeable, porous, mesh or grid-like material and can include copper, copper alloys, or copper mesh material. Other metallic materials with known antimicrobial properties may also be used, such as silver or zinc, or alloys thereof. In an embodiment, filters can include at least one layer of copper oxide-impregnated polypropylene fibers. Filters 4, 18 can also include at least one layer of a woven or non-woven material that itself acts as a filter or is treated to filter. The first and second filters 4, 18 would allow the patient 24 to breathe naturally, while keeping airborne particles from the air external to the mask 2. The size of the filter is such that it prevents known viruses and microbials from transmitting into the air external to the mask. In the illustrated embodiments, the first and second filters 4, 18 are sized to prevent known viruses and microbials, including human coronavirus particles from entering the air external to the mask. This prevents medical professionals from inhaling said viruses and microbials during the course of an endoscopic procedure. In the illustrated embodiment, the first and second filters 4, 18 are in the filter size range of 70 microns-100 microns. In embodiments where the mask 2 is reusable, the at least one filter may be replaced with a new filter for each use.

The mask can be manufactured and assembled by means known in the art. Clear flexible plastic materials may be used for creating the mask with cut outs for the filters and central opening. The seals, filters and rim material may be secured to the mask by means such as adhesives or other bonding methods.

Modifications in addition to those described about may be made to the structures and techniques described herein without departing from the spirit and scope of the disclosure.

What is claimed is:

1. A mask for use in endoscopic procedures comprising:
a unitary concave and bowl-shaped body portion having an inner surface and an outer surface;
said body having a first opening for receiving an instrument, said first opening in said body defined by an edge, said outer surface radially extending from said edge with a unitary curved wall having a peak and extending from said peak to a rim, said rim defining a second opening;
said second opening wider than said first opening and having a diameter of sufficient width to cover a patient's nose and open mouth;
at least one filter positioned on said body portion to permit air to pass through said body portion;
a seal positioned around or over said first opening to secure said instrument when received through said first opening.

2. The mask of claim 1, wherein the second opening ranges in size to fit a variety of patients.

3. The mask of claim 1, wherein the first opening may range in size from 2 mm-12 mm to accommodate an endoscope or instrument.

4. The mask of claim 1, wherein the first opening is centrally located.

5. The mask of claim 1, wherein the seal positioned around the first opening seals the endoscope hermetically.

6. The mask of claim 1, wherein the seal is flexible so as to maintain the hermetic seal of the instrument while moving during a procedure.

7. The mask of claim 1, wherein the at least one filter is positioned on the inner surface of the mask.

8. The mask of claim 1, wherein the at least one filter is positioned on the mask for filtration of microdroplets from a respiratory process of a patient.

9. The mask of claim 1, wherein the at least one filter is of a size to prevent known viruses and microbials from transmitting into external air outside the mask.

10. The mask of claim 1, wherein the at least one filter is of a size to prevent human coronavirus particles from transmitting into external air outside the mask.

11. The mask of claim 1, wherein the at least one filter has a size range of 70 microns-100 microns for filtration.

12. The mask of claim 1, wherein the at least one filter is made of a porous, grid-like material.

13. The mask of claim 1, wherein one or more hermetically sealed openings are included on the mask for surgical instruments in addition to a primary instrument.

14. The mask of claim 1, wherein the mask is secured over a patient's nose and mouth during use, with the inner surface of the mask positioned toward the patient.

15. The mask of claim 1, wherein the mask is secured to a patient's head by use of a strap.

16. The mask of claim 1, wherein the bowl-shaped body portion is made of a transparent material.

17. The mask of claim 1, wherein the rim is covered by a soft material capable of molding to a patient's face.

18. The mask of claim 1, wherein the bowl-shaped body portion is made of a hypoallergenic material.

19. The mask of claim 1, wherein the mask is capable of withstanding repeated sterilization processes.

20. The mask of claim 1, wherein the mask is single-use and disposable.

21. The mask of claim 1, wherein said at least one filter includes copper, copper alloys, or copper mesh material.
